(19) United States
(12) Patent Application Publication
(10) Pub. No.: US 2023/0207072 A1
(43) Pub. Date: Jun. 29, 2023

(54) SYSTEM AND METHOD FOR MANAGING CLINICAL TRIAL DATA USING BLOCKCHAIN NETWORK SCHEME

(71) Applicant: Electronics and Telecommunications Research Institute, Daejeon (KR)

(72) Inventors: Bumhwi KIM, Daejeon (KR); Kyu Hyung KIM, Daejeon (KR)

(73) Assignee: Electronics and Telecommunications Research Institute, Daejeon (KR)

(21) Appl. No.: 17/974,959

(22) Filed: Oct. 27, 2022

(30) Foreign Application Priority Data
Dec. 28, 2021 (KR) 10-2021-0190051

(51) Int. Cl. G16H 10/20; H04L 9/32

(52) U.S. Cl. CPC G16H 10/20; H04L 9/3242

(57) ABSTRACT

Provided are a system and method for managing clinical trial data using a blockchain network scheme which allow hospitals, a contract research organization (CRO) group, and a blockchain network admin entity to share clinical trial data resulting from a clinical trial in real time on the basis of a blockchain network but forbid a hospital from accessing data generated from a plurality of other clinical hospitals in one clinical trial, encode and encrypt recorded information such that interpretation is not possible even through direct blockchain network access, and thereby ensure the integrity of clinical trial result data and enhance anonymity and security thereof.

### CROSS-REFERENCE TO RELATED APPLICATION

This application claims priority to and the benefit of Korean Patent Application No. 10-2021-0190051 filed on Dec. 28, 2021, the disclosure of which is incorporated herein by reference in its entirety.

### BACKGROUND

### 1. Field of the Invention

The present invention relates to a system and method for managing clinical trial data using a blockchain network scheme.

### 2. Discussion of Related Art

Currently, information generated from a clinical trial is recorded online. However, not all information is recorded at distributed locations, and the data is recorded in a centralized manner. For this reason, it is difficult to flexibly cope with situations such as a network error and a blackout. Also, when there is data leakage, such as a third party reading data through hacking, or data forgery, it is difficult to ensure the reliability of a clinical trial, which may result in a greater problem.

Accordingly, it is necessary to develop a technology for preventing data forgery by enabling a plurality of entities to share clinical trial data in real time using a blockchain network scheme and for preventing a third party from identifying unexpectedly leaked data by encrypting clinical trial data.

### SUMMARY OF THE INVENTION

The present invention is directed to providing a system and method for managing clinical trial data using a blockchain network scheme which allow hospitals, a contract research organization (CRO) group, and a blockchain network admin entity to share clinical trial data resulting from a clinical trial in real time on the basis of a blockchain network but forbid a hospital from accessing data generated from a plurality of other clinical hospitals in one clinical trial, encode and encrypt recorded information such that interpretation is not possible even through direct blockchain network access, and thereby ensure the integrity of clinical trial result data and enhance anonymity and security thereof.

According to an aspect of the present invention, there is provided a system for managing clinical trial data using a blockchain network scheme, the system including a RESTful application programming interface (API) router connected to a hospital terminal and a smart pillbox terminal through a web server, a blockchain platform configured to define group-specific attributes for a CRO group for managing a clinical trial, a hospital group for carrying out the clinical trial, and an admin group for administering a blockchain network, a database (DB) configured to receive test information of clinical trial subjects from the hospital terminal, receive medication information and vital sign information of the clinical trial subjects from the smart pillbox terminal, and encrypt and then store the test information, the medication information, and the vital sign information, and a blockchain network admin part configured to separately give authority to access the blockchain network to the groups and provide the test information, the medication information, and the vital sign information of the clinical trial subjects stored in the DB to be viewed or read through group-specific management terminals but forbid other groups from viewing or reading the test information, the medication information, and the vital sign information of the clinical trial subjects.

The RESTful API router may be linked with the blockchain platform through Google remote procedure calls (gRPC) and a hypertext transfer protocol (HTTP).

Each of the CRO group, the hospital group, and the manager group may include one or more peer domains in which a commit function and an endorsing function are performed at a blockchain node, an orderer configured to align transactions generated from peers, a private data collection domain including personal information of each of the clinical trial subjects, a state DB domain including updates and reflections of information in the blockchain network, a fabric certificate authority (CA) domain including group certifications, and a membership service provider (MSP) domain for the group.

After the test information, the medication information, and the vital sign information of the clinical trial subjects are received, the DB may substitute detailed test item names, such as test information, medication information, and vital sign information, with code through aliasing, encrypt the code on the basis of a Base64 encryption scheme, and store the encrypted code.

The DB may encrypt identification (ID) information given to each of the clinical trial subjects on the basis of a SHA-256 hash encryption scheme and store the encrypted ID information.

The DB may generate key data including the test information encrypted on the basis of the Base64 encryption scheme and the ID information encrypted on the basis of the SHA-256 hash encryption scheme and then store the key data.

When viewing or reading information of the key data is requested through the group-specific management terminals, the blockchain network admin part may decrypt and provide the key data to the group-specific management terminals with the test information and the ID information included in the key data prevented from being decrypted.

The blockchain network admin part may separately give the authority to access the blockchain network to the groups on the basis of a public key infrastructure (PKI)-based MSP.

The MSP may be defined to include organization information, peer information, orderer information, admin information, user role information, user affiliation information, and user authority information.

The blockchain network admin part may load a user authentication file and an access configuration file into a file system in the blockchain network.

The access configuration file may include organization information, peer information, and transport layer security (TLS) certificate information.

The DB may repeatedly receive the medication information and the vital sign information including electrocardiogram (ECG) information, blood pressure information, and blood sugar information of the clinical trial subjects from the smart pillbox terminal and datafy the medication information and the vital sign information.

According to another aspect of the present invention, there is provided a system for managing clinical trial data using a blockchain network scheme in association with a remote health diagnostics and monitoring system (RHDMS), the system including a blockchain platform configured to define group-specific attributes for a CRO group for managing a clinical trial, a hospital group for carrying out the clinical trial, and an admin group for administering a blockchain network and separately giving authority to access the blockchain network to the groups, a RESTful API router connected to a hospital terminal and a smart pillbox terminal through a web server and linked with the blockchain platform through gRPC and an HTTP, a DB configured to receive test information of clinical trial subjects from the hospital terminal, receive medication information and vital sign information of the clinical trial subjects from the smart pillbox terminal, and encrypt and then store the test information, the medication information, and the vital sign information, and a blockchain network admin part configured to separately give the authority to access the blockchain network to the groups and provide the test information, the medication information, and the vital sign information of the clinical trial subjects stored in the DB to be viewed or read through group-specific management terminals but forbid other groups from viewing or reading the test information, the medication information, and the vital sign information of the clinical trial subjects.

After the test information, the medication information, and the vital sign information of the clinical trial subjects are received, the DB may substitute detailed test item names, such as test information, medication information, and vital sign information, with code through aliasing, encrypt the code on the basis of a Base64 encryption scheme, and store the encrypted code, and may also encrypt ID information separately given to the clinical trial subjects on the basis of SHA-256 hash encryption scheme and then store the encrypted ID information.

According to another aspect of the present invention, there is provided a method of managing clinical trial data using a blockchain network scheme, the method including connecting a RESTful API router to a hospital terminal and a smart pillbox terminal through a web server, defining, by a blockchain platform, group-specific attributes for a CRO group for managing a clinical trial, a hospital group for carrying out the clinical trial, and an admin group for administering a blockchain network, receiving, by a DB, test information of clinical trial subjects from the hospital terminal, receiving medication information and vital sign information of the clinical trial subjects from the smart pillbox terminal, and encrypting and then storing the test information, the medication information, and the vital sign information, and separately giving, by a blockchain network admin part, authority to access the blockchain network to the groups and providing the test information, the medication information, and the vital sign information of the clinical trial subjects stored in the DB to be viewed or read through group-specific management terminals but forbidding other groups from viewing or reading the test information, the medication information, and the vital sign information of the clinical trial subjects.

### BRIEF DESCRIPTION OF THE DRAWINGS

The above and other objects, features and advantages of the present invention will become more apparent to those of ordinary skill in the art by describing exemplary embodiments thereof in detail with reference to the accompanying drawings, in which:

FIG. 1 is a block diagram of a system (100) for managing clinical trial data using a blockchain network scheme according to the present invention;

FIGS. 2A to 2D are diagrams illustrating the concept of the system (100) for managing clinical trial data using a blockchain network scheme shown in FIG. 1;

FIGS. 3A to 3C are diagrams illustrating a blockchain platform (120) shown in FIGS. 2A to 2D in more detail; and

FIGS. 4A and 4B are set of flowcharts sequentially illustrating processes of reading and writing blockchain network data through the system (100) for managing clinical trial data using a blockchain network scheme in association with a remote health diagnostics and monitoring system (RHDMS).

### DETAILED DESCRIPTION OF EXEMPLARY EMBODIMENTS

Hereinafter, exemplary embodiments will be proposed to help with understanding of the present invention. However, the following embodiments are merely provided to facilitate understanding of the present invention, and the present invention is not limited thereto.

The present invention relates to a system and method for managing clinical trial data using a blockchain network scheme, and more particularly, to a system and method for managing clinical trial data using a blockchain network scheme which allow hospitals, a contract research organization (CRO) group, and a blockchain network admin entity to share clinical trial data resulting from a clinical trial in real time on the basis of a blockchain network but forbid a hospital from accessing data generated from a plurality of other clinical hospitals in one clinical trial, encode and encrypt recorded information such that interpretation is not possible even through direct blockchain network access, and thereby ensure the integrity of clinical trial result data and enhance anonymity and security thereof.

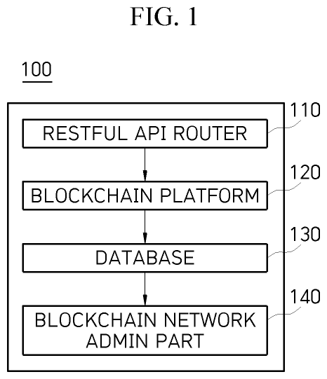

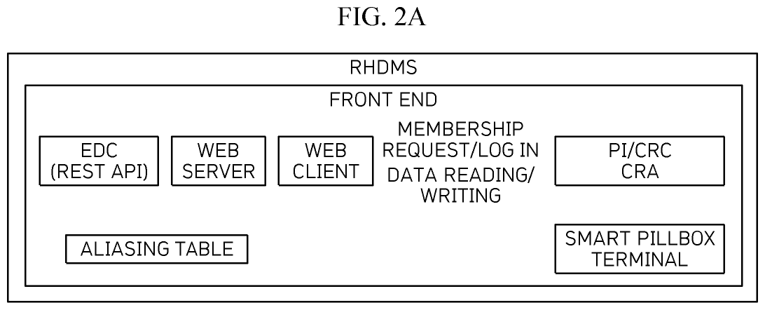

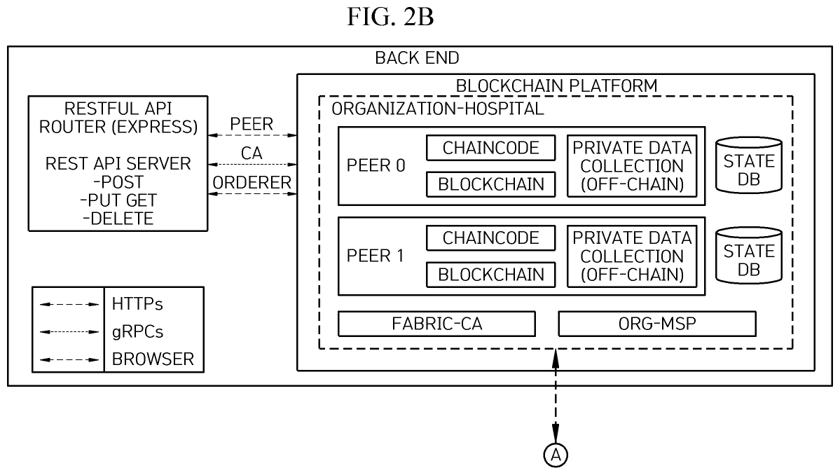

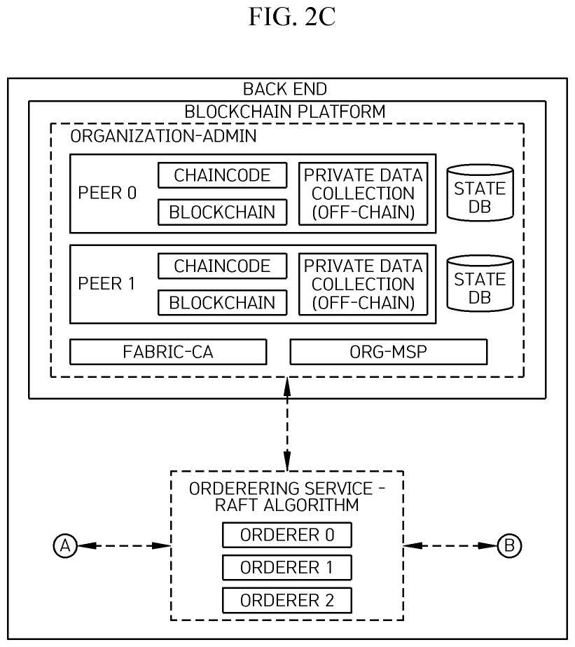

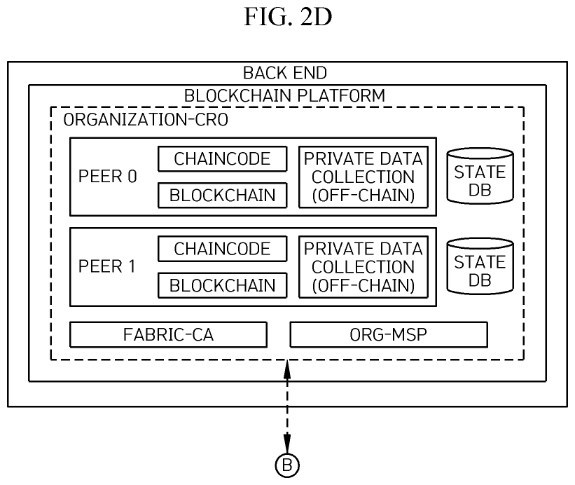

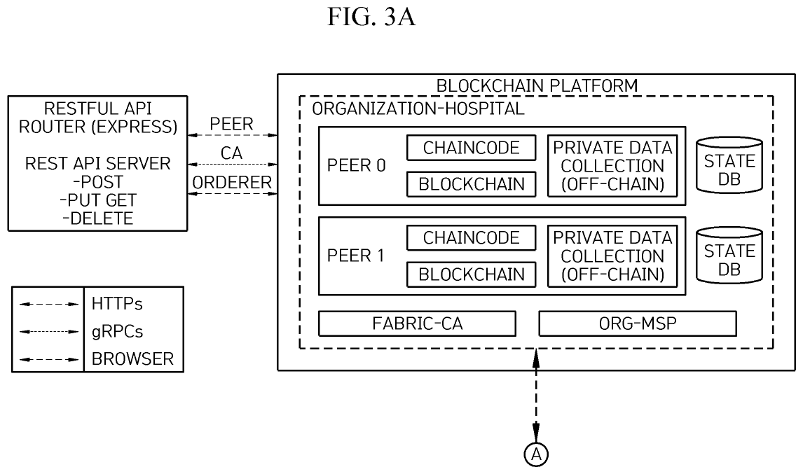

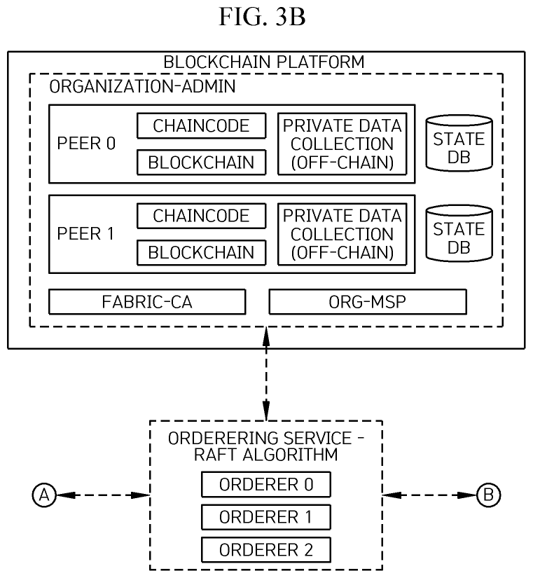

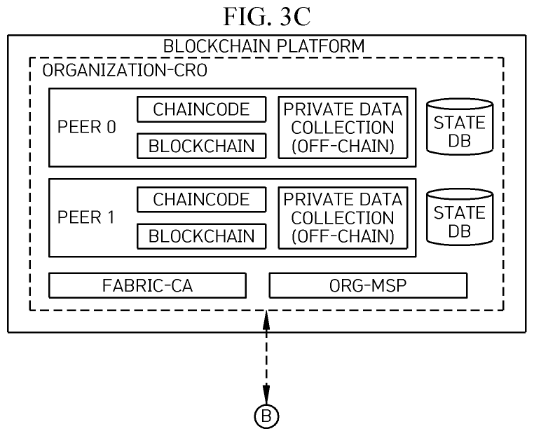

FIG. 1 is a block diagram of a system 100 for managing clinical trial data using a blockchain network scheme according to the present invention, FIGS. 2A to 2D are diagrams illustrating the concept of the system 100 for managing clinical trial data using a blockchain network scheme shown in FIG. 1, and FIGS. 3A to 3C is a diagram illustrating a blockchain platform 120 shown in FIGS. 2A to 2D in more detail.

The term “clinical trial data” used herein may refer to data that is generated when a clinical trial subject goes to a hospital and undergoes a test and data that is generated when a clinical trial subject takes a medication directly through a smart pillbox at his or her house.

Referring to FIGS. 1 to 3, the system 100 for managing clinical trial data using a blockchain network scheme according to the present invention may include a RESTful application programming interface (API) router 110, a blockchain platform 120, a database (DB) 130, and a blockchain network admin part 140.

The RESTful API router 110 may be connected to a hospital terminal and a smart pillbox through a web server and may also be linked with the blockchain platform 120 through Google remote procedure calls (gRPC) and the hypertext transfer protocol (HTTP). Also, the RESTful API router 110 may have a structure that is linked with an electronic data capture (EDC), a web server, and the smart pillbox.

The blockchain platform 120 defines a CRO group for managing a clinical trial, a hospital group for carrying out the clinical trial, and an admin group for administering a blockchain network and also defines group-specific attributes. More specifically, the blockchain platform 120 includes an orderer group, the CRO group, the hospital group, and the admin group, and each of the groups which operate on the basis of a raft consensus algorithm, and each of the groups may include one or more peer domains (e.g., Peer0 and Peer1) in which a commit function and an endorsing function are performed at a blockchain node, a private data collection domain which includes personal information of each clinical trial subject, a state DB domain which updates and reflects information in the blockchain network in the latest state, a fabric certificate authority (CA) domain including group certifications for group authentication, and a membership service provider (MSP) domain for each of the groups. Referring to FIGS. 3A to 3C, the blockchain platform 120 includes models that are types of information on modules of Hyperledger fabric for constructing a blockchain network, a controller that manages resources for the fabric and a user, a generator that generates fabric resources, a factory that creates each node using resources, various external tools that help with use of fabric, etc., and each function may be implemented through a command-line interface (CLI) commander and an API server.

The DB 130 may receive test information of a clinical trial subject from the hospital terminal, receive medication information and vital sign information of the clinical trial subject from a smart pillbox terminal, and encrypt and then store the test information, the medication information, and the vital sign information.

More specifically, according to the present invention, a remote health diagnostics and monitoring system (RHDMS), which is a smart clinical system, manages various data stored in the blockchain network through encoding. After the test information, the medication information, and the vital sign information of the clinical trial subject are received, the DB 130 may encrypt test date information, test name information, and test item information included in the test information on the basis of a recoverable Base64 encryption scheme, and store the encrypted information. Also, the database 130 may encrypt identification (ID) information given to each clinical trial subject on the basis of an unrecoverable SHA-256 hash encryption scheme and store the encrypted ID information.

When detailed test item names, such as test information, medication information, and vital sign information, are substituted with code through aliasing, test item names are also encoded and used with substitutions so that the detailed test item names cannot be recognized by a third party.

As an example,

Blood pressure test:

Systolic blood pressure (SBP): 120,

Diastolic blood pressure (DBP): 80, and

Pulse: 60

are substituted with

0001:

01:120,

02:80, and

03:60.

0001(General classification: hospital/pillbox)_0001(Medium classification: test name)_01(Detailed classification: detailed test item name)_01(Option) order: in the case of an item occurring several times a day) is substituted with 000000010101.

The DB 130 generates and stores key data including the test information encrypted on the basis of the Base64 encryption scheme and the ID information encrypted on the basis of the SHA-256 hash encryption scheme and then stores the key data.

Meanwhile, key data that is finally stored in a DB of the blockchain network may be, for example, “SHA-256(ID information)_date_code,” and a value may be generated in the form “Base64(test information or test item information).”

Also, the DB 130 separately applies branch encryption to the blockchain, and in this case, a key-value DB is used for storage in the blockchain. This may be divided into an operation of generating a key and an operation of encrypting a value.

The key may be stored in order of date_code_name, and SHA-256 encryption may be performed on the name among them. Code related thereto is as follows.

### Code Example

Test item code: {
Detailed item code: value,
Detailed item code: value,
Detailed item code: value,
...
}

Referring to the code, the code substitutes for whether a hospital or a pillbox is used + test item names. Item names in text are substituted with numeric code so that the numeric code cannot be recognized by third parties. In particular, a test-code matching table is managed by an RHDMS server.

Finally, a key is generated as date_aliasing(code)_sha256(ID), and as for detailed test items in test items which are data, names thereof are substituted through aliasing and encrypted on the basis of the Base64 encryption scheme.

The blockchain network admin part 140 separately gives authority to access the blockchain network to the groups and provides the test information, the medication information, and the vital sign information of the clinical trial subject stored in the DB 130 to be viewed or read through group-specific management terminals. However, the blockchain network admin part 140 forbids other groups from viewing or reading the test information, the medication information, and the vital sign information of the clinical trial subject.

More specifically, when viewing or reading information of the previously generated key data is requested through the group-specific management terminals, the blockchain network admin part 140 decrypts and provides the previously generated key data to the group-specific management terminals. In this process, the blockchain network admin part 140 forbids a management terminal that is not allowed to read the key data from checking corresponding content by forbidding decoding of the test information and the ID information.

When a clinical trial subject goes to a hospital in person and undergoes a clinical test, different tests are carried out depending on the types of clinical trials. In this case, results corresponding to the types of clinical tests may be encrypted through the DB 130 and recorded in the blockchain network. In the case of the clinical trial subject's own house, the vital sign information including administration information (whether the subject takes a medication, the number of administrations, etc.), electrocardiogram (ECG) information, blood pressure information, and blood sugar information of the clinical trial subject may be repeatedly received at regular intervals from a smart pillbox terminal and datafied, which may be recorded in the blockchain network.

A process in which the above-described clinical trial data of a clinical trial subject is recorded on a blockchain network will be described below.

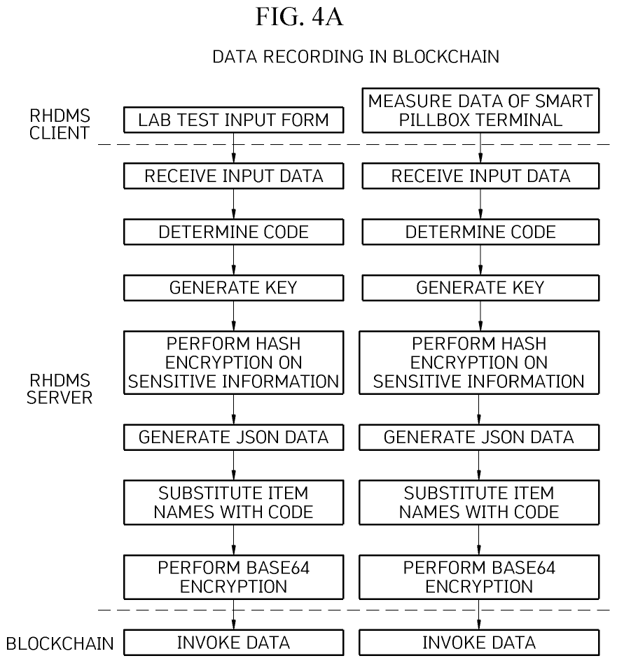

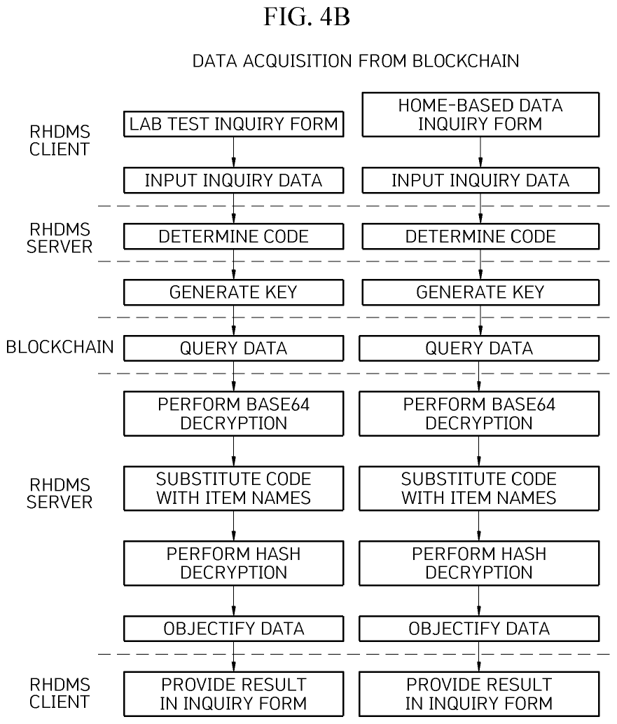

FIGS. 4A and 4B are set of flowcharts sequentially illustrating processes of reading and writing blockchain network data through the system 100 for managing clinical trial data using a blockchain network scheme in association with an RHDMS.

FIGS. 4A and 4B illustrates the processes of reading and writing clinical trial data from and on a blockchain network through the system 100 for managing clinical trial data using a blockchain network scheme in association with an RHDMS.

First, referring to the process of recording clinical trial data on a blockchain, a client end of an RHDMS submits a lab test input form or measures data of a smart pillbox, and a server end of the RHDMS receives input data, determines code, generates a key, performs hash encryption on sensitive information, such as personal information, generates JavaScript Object Notation (JSON) data, substitutes clinical test item names with code, and then encrypts the code using a Base64 encryption scheme.

Subsequently, referring to the process of acquiring clinical trial data from a blockchain, a client end of the RHDMS submits a lab test inquiry form or a home-based data inquiry form for a clinical trial subject to input corresponding inquiry information.

The server end of the RHDMS generates a key of the input inquiry information, and a blockchain end generates query data on the basis of the key. The server end of the RHDMS decrypts the query data using the Base64 encryption scheme, separately substitutes code according to clinical test items, and performs hash decryption to objectify data. This result is output as an inquiry form by the client end of the RHDMS.

Meanwhile, in the present invention, a blockchain network admin does not directly have a part in data in the blockchain network, and the blockchain network admin part manages authority and authentication of a user, a member, a channel, and an organization on the basis of a public key infrastructure (PKI)-based MSP file.

The MSP file defines roles, affiliations, authority, etc. of organizations, peers, orderers, an admin, and users. Accordingly, when an MSP file is generated and distributed to the file system of each node, the blockchain network is accessible.

Also, when a user authentication file (an identity key file) and an access configuration file are distributed and loaded into the file system of a corresponding node, fabric is accessible.

The access configuration file is defined to include organization information, peer information, orderer information, admin information, user role information, user affiliation information, and user authority information.

Meanwhile, a node having the MSP file in the blockchain network according to the present invention may read or write data from or in the blockchain network using inter-peer chaincode in an application through a fabric-software development kit (SDK).

To read or write data from or in the blockchain network, it is necessary to install chaincode. Accordingly, a chaincode installation process, such as installation, virtualization, and upgrade of chaincode, is performed by a blockchain network admin terminal, and installation is performed in group-specific peers. Subsequently, a chaincode container for the chaincode is created, and thus it is possible to read or write data from or in the blockchain network.

According to the present invention, hospitals, a CRO group, and a blockchain network admin entity are allowed to share clinical trial data resulting from a clinical trial in real time on the basis of a blockchain network, but a hospital is forbidden from accessing data generated from a plurality of other clinical hospitals in one clinical trial, and recorded information is encoded and encrypted such that interpretation is not possible even through direct blockchain network access. Therefore, it is possible to ensure the integrity of clinical trial result data and enhance anonymity and security thereof.

Although the present invention has been described above with reference to exemplary embodiments, those of ordinary skill in the art will appreciate that various modifications and alterations can be made without departing from the spirit and scope of the present invention set forth in the following claims.

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The components described in the example embodiments may be implemented by hardware components including, for example, at least one digital signal processor (DSP), a processor, a controller, an application-specific integrated circuit (ASIC), a programmable logic element, such as an FPGA, other electronic devices, or combinations thereof. At least some of the functions or the processes described in the example embodiments may be implemented by software, and the software may be recorded on a recording medium. The components, the functions, and the processes described in the example embodiments may be implemented by a combination of hardware and software.

The method according to example embodiments may be embodied as a program that is executable by a computer, and may be implemented as various recording media such as a magnetic storage medium, an optical reading medium, and a digital storage medium.

Various techniques described herein may be implemented as digital electronic circuitry, or as computer hardware, firmware, software, or combinations thereof. The techniques may be implemented as a computer program product, i.e., a computer program tangibly embodied in an information carrier, e.g., in a machine-readable storage device (for example, a computer-readable medium) or in a propagated signal for processing by, or to control an operation of a data processing apparatus, e.g., a programmable processor, a computer, or multiple computers. A computer program(s) may be written in any form of a programming language, including compiled or interpreted languages and may be deployed in any form including a stand-alone program or a module, a component, a subroutine, or other units suitable for use in a computing environment. A computer program may be deployed to be executed on one computer or on multiple computers at one site or distributed across multiple sites and interconnected by a communication network.

Processors suitable for execution of a computer program include, by way of example, both general and special purpose microprocessors, and any one or more processors of any kind of digital computer. Generally, a processor will receive instructions and data from a read-only memory or a random access memory or both. Elements of a computer may include at least one processor to execute instructions and one or more memory devices to store instructions and data. Generally, a computer will also include or be coupled to receive data from, transfer data to, or perform both on one or more mass storage devices to store data, e.g., magnetic, magneto-optical disks, or optical disks. Examples of information carriers suitable for embodying computer program instructions and data include semiconductor memory devices, for example, magnetic media such as a hard disk, a floppy disk, and a magnetic tape, optical media such as a compact disk read only memory (CD-ROM), a digital video disk (DVD), etc. and magneto-optical media such as a floptical disk, and a read only memory (ROM), a random access memory (RAM), a flash memory, an erasable programmable ROM (EPROM), and an electrically erasable programmable ROM (EEPROM) and any other known computer readable medium. A processor and a memory may be supplemented by, or integrated into, a special purpose logic circuit.

The processor may run an operating system (OS) and one or more software applications that run on the OS. The processor device also may access, store, manipulate, process, and create data in response to execution of the software. For purpose of simplicity, the description of a processor device is used as singular; however, one skilled in the art will be appreciated that a processor device may include multiple processing elements and/or multiple types of processing elements. For example, a processor device may include multiple processors or a processor and a controller. In addition, different processing configurations are possible, such as parallel processors.

Also, non-transitory computer-readable media may be any available media that may be accessed by a computer, and may include both computer storage media and transmission media.

The present specification includes details of a number of specific implements, but it should be understood that the details do not limit any invention or what is claimable in the specification but rather describe features of the specific example embodiment. Features described in the specification in the context of individual example embodiments may be implemented as a combination in a single example embodiment. In contrast, various features described in the specification in the context of a single example embodiment may be implemented in multiple example embodiments individually or in an appropriate sub-combination. Furthermore, the features may operate in a specific combination and may be initially described as claimed in the combination, but one or more features may be excluded from the claimed combination in some cases, and the claimed combination may be changed into a sub-combination or a modification of a sub-combination.

Similarly, even though operations are described in a specific order on the drawings, it should not be understood as the operations needing to be performed in the specific order or in sequence to obtain desired results or as all the operations needing to be performed. In a specific case, multitasking and parallel processing may be advantageous. In addition, it should not be understood as requiring a separation of various apparatus components in the above described example embodiments in all example embodiments, and it should be understood that the above-described program components and apparatuses may be incorporated into a single software product or may be packaged in multiple software products.

It should be understood that the example embodiments disclosed herein are merely illustrative and are not intended to limit the scope of the invention. It will be apparent to one of ordinary skill in the art that various modifications of the example embodiments may be made without departing from the spirit and scope of the claims and their equivalents.

What is claimed is:

1. A system for managing clinical trial data using a blockchain network scheme, the system comprising:
a RESTful application programming interface (API) router connected to a hospital terminal and a smart pillbox terminal through a web server;
a blockchain platform configured to define group-specific attributes for a contract research organization (CRO) group for managing a clinical trial, a hospital group for carrying out the clinical trial, and an admin group for administering a blockchain network;
a database (DB) configured to receive test information of clinical trial subjects from the hospital terminal, receive medication information and vital sign information of the clinical trial subjects from the smart pillbox terminal, and encrypt and then store the test information, the medication information, and the vital sign information; and
a blockchain network admin part configured to separately give authority to access the blockchain network to the groups and provide the test information, the medication information, and the vital sign information of the clinical trial subjects stored in the DB to be viewed or read through group-specific management terminals but forbid other groups from viewing or reading the test information, the medication information, and the vital sign information of the clinical trial subjects.

2. The system of claim 1, wherein the RESTful API router is linked with the blockchain platform through Google remote procedure calls (gRPC) and a hypertext transfer protocol (HTTP).

3. The system of claim 1, wherein each of the CRO group, the hospital group, and the manager group includes:
one or more peer domains in which a commit function and an endorsing function are performed at a blockchain node;
a private data collection domain including personal information of each of the clinical trial subjects;
a state DB domain including updates and reflections of information in the blockchain network;
a fabric certificate authority (CA) domain including group certifications; and
a membership service provider (MSP) domain for the group.

4. The system of claim 1, wherein, after the test information, the medication information, and the vital sign information of the clinical trial subjects are received, the DB substitutes detailed test item names with code through aliasing, encrypts the code on the basis of a Base64 encryption scheme, and stores the encrypted code.

5. The system of claim 4, wherein the DB encrypts identification (ID) information given to each of the clinical trial subjects on the basis of a SHA-256 hash encryption scheme and stores the encrypted ID information.

6. The system of claim 5, wherein the DB generates key data including the test information encrypted on the basis of the Base64 encryption scheme and the ID information encrypted on the basis of the SHA-256 hash encryption scheme and then stores the key data.

7. The system of claim 6, wherein, when viewing or reading information of the key data is requested through the group-specific management terminals, the blockchain network admin part decrypts and provides the key data to the group-specific management terminals with the test information and the ID information included in the key data prevented from being decrypted.

8. The system of claim 1, wherein the blockchain network admin part separately gives the authority to access the blockchain network to the groups on the basis of a public key infrastructure (PKI)-based membership service provider (MSP).

9. The system of claim 8, wherein the MSP is defined to include organization information, peer information, orderer information, admin information, user role information, user affiliation information, and user authority information.

10. The system of claim 8, wherein the blockchain network admin part loads a user authentication file and an access configuration file into a file system in the blockchain network.

11. The system of claim 10, wherein the access configuration file includes organization information, peer information, and transport layer security (TLS) certificate information.

12. The system of claim 1, wherein the DB repeatedly receives the medication information and the vital sign information including electrocardiogram (ECG) information, blood pressure information, and blood sugar information of the clinical trial subjects from the smart pillbox terminal and datafies the medication information and the vital sign information.

13. A system for managing clinical trial data using a blockchain network scheme in association with a remote health diagnostics and monitoring system (RHDMS), the system comprising:
a blockchain platform configured to define group-specific attributes for a contract research organization (CRO) group for managing a clinical trial, a hospital group for carrying out the clinical trial, and an admin group for administering a blockchain network and separately giving authority to access the blockchain network to the groups;
a RESTful application programming interface (API) router connected to a hospital terminal and a smart pillbox terminal through a web server and linked with the blockchain platform through Google remote procedure calls (gRPC) and a hypertext transfer protocol (HTTP);
a database (DB) configured to receive test information of clinical trial subjects from the hospital terminal, receive medication information and vital sign information of the clinical trial subjects from the smart pillbox terminal, and encrypt and then store the test information, the medication information, and the vital sign information; and
a blockchain network admin part configured to separately give the authority to access the blockchain network to the groups and provide the test information, the medication information, and the vital sign information of the clinical trial subjects stored in the DB to be viewed or read through group-specific management terminals but forbid other groups from viewing or reading the test information, the medication information, and the vital sign information of the clinical trial subjects.

14. The system of claim 13, wherein, after the test information, the medication information, and the vital sign information of the clinical trial subjects are received, the DB substitutes detailed test item names with code through aliasing, encrypts the code on the basis of a Base64 encryption scheme, and stores the encrypted code, and
the DB encrypts identification (ID) information separately given to the clinical trial subjects on the basis of SHA-256 hash encryption scheme and then stores the encrypted ID information.

15. A method of managing clinical trial data using a blockchain network scheme, the method comprising:
connecting a RESTful application programming interface (API) router to a hospital terminal and a smart pillbox terminal through a web server;
defining, by a blockchain platform, group-specific attributes for a contract research organization (CRO) group for managing a clinical trial, a hospital group for carrying out the clinical trial, and an admin group for administering a blockchain network;
receiving, by a database (DB), test information of clinical trial subjects from the hospital terminal, receiving medication information and vital sign information of the clinical trial subjects from the smart pillbox terminal, and encrypting and then storing the test information, the medication information, and the vital sign information; and
separately giving, by a blockchain network admin part, authority to access the blockchain network to the groups and providing the test information, the medication information, and the vital sign information of the clinical trial subjects stored in the DB to be viewed or read through group-specific management terminals but forbidding other groups from viewing or reading the test information, the medication information, and the vital sign information of the clinical trial subjects.
